(19) United States
(12) Patent Application Publication
(10) Pub. No.: US 2024/0091508 A1
(43) Pub. Date: Mar. 21, 2024

(54) MEDICAL GUIDEWIRE DEVICES AND SYSTEMS WITH CONTROLLABLE FEATURES AND RELATED METHODS

(71) Applicants: Boston Scientific Medical Device Limited, Galway (IE); Boston Scientific Scimed, Inc., Maple Grove, MN (US)

(72) Inventors: Deepak Kumar SHARMA, Muzaffarnagar (IN); Charles GIBSON, Malden, MA (US); James SCUTTI, Norwell, MA (US); Sharath Kumar G., Kanakapura (IN); Juan Pablo Ortiz GARCIA, San Isidro (CR)

(73) Assignees: Boston Scientific Medical Device Limited, Galway Park (IE); Boston Scientific Scimed, Inc., Maple Grove, MN (US)

(21) Appl. No.: 18/470,628

(22) Filed: Sep. 20, 2023

Related U.S. Application Data
(60) Provisional application No. 63/408,705, filed on Sep. 21, 2022.

(51) Int. Cl. A61M 25/09

(52) U.S. Cl. CPC A61M 25/0905; A61M 25/09; A61M 2025/09125

(57) ABSTRACT

A guidewire assembly may include a handle assembly including: a handle body; a shaft extending through the handle body; and an actuator. The guidewire assembly may further include a tube extending distally from the handle assembly and including a proximal portion and a distal portion; and an interior body extending from the handle assembly through the tube to the distal portion of the tube, wherein the interior body is coupled to the shaft. The shaft may be configured to move longitudinally through the handle body as the actuator is rotated to move interior body longitudinally through the tube.

### CROSS-REFERENCE TO RELATED APPLICATIONS

This application claims the benefit of priority of U.S. Provisional Patent Application No. 63/408,705, filed on Sep. 21, 2022, the entirety of which is incorporated herein by reference.

### TECHNICAL FIELD

Various aspects of the present disclosure relate generally to medical systems, devices, and related methods. More specifically, the present disclosure relates to medical systems, devices, and methods for positioning one or more medical devices within a body lumen.

### BACKGROUND

Medical procedures that involve navigating to a site within the body often require a medical guidewire. Guidewires are used in numerous catheterization procedures and other medical procedures as an aid to placement of a catheter or other device at a selected site within the human body. Guidewires may be advanced under direct visualization through an endoscope, with or without fluoroscopy. Typically, guidewires are lubricious to facilitate easy movement in small tubular body lumens. The low friction from the lubrication facilitates force transmission and fine movement when small forces are exerted on the proximal end of the wire. Once the guidewire is placed proximate to target anatomy, maintenance of the guidewire position is desirable for the safety and efficiency of a procedure. Navigation of a guidewire often requires movement through complex, tortuous anatomies. In some examples, a user may have difficulty locating target anatomy within a body lumen to begin a procedure because of limitations on the guidewire's ability to move within the body lumen. Difficulties in guidewire movement may prolong a procedure and/or increase the likelihood of a complication and/or a failed procedure.

The systems, devices, and methods of the current disclosure may rectify some of the deficiencies described above, and/or may address other aspects of the art.

### SUMMARY

Examples of the present disclosure relate to, among other things, medical systems, devices, and methods. Each of the examples disclosed herein may include one or more of the features described in connection with any of the other disclosed examples.

According to one aspect, a guidewire assembly may include a handle assembly including: a handle body; a shaft extending through the handle body; and an actuator. The guidewire assembly may further include a tube extending distally from the handle assembly and including a proximal portion and a distal portion; and an interior body extending from the handle assembly through the tube to the distal portion of the tube, wherein the interior body is coupled to the shaft. The shaft may be configured to move longitudinally through the handle body as the actuator is rotated to move interior body longitudinally through the tube.

In other aspects, the guidewire assembly may include one or more of the following features. A distal portion of the interior body may be shape-memory material. The distal portion of the interior body may be U-shaped, S-shaped, helical shaped, spiral shaped, and/or includes a one hundred and eighty degree curve in the interior body. The proximal portion of the tube may include a laser cut pattern. The tube may be coupled to the handle assembly at a distal end of the handle assembly. The tube may be removably coupled to the distal end of the handle assembly via a distal cap and a distal collet. The actuator may be cylindrical and extend longitudinally through a central longitudinal axis of the handle body, and the shaft may extend longitudinally through a lumen of the actuator. The handle body may include a cylindrical proximal portion, a distal portion spaced from the proximal portion, and a pair of frame arms coupled to the distal portion and the proximal portion. A distal tip of interior body may be coupled to a distal tip portion of the tube. The actuator may include a circular protrusion received by a recess of each of the pair of frame arms. The interior body may be removably coupled to a proximal end of the shaft via a proximal collet and a proximal cap. The actuator may include a helical recess configured to receive a helical protrusion of the shaft. The actuator may be configured to move the shaft proximally or distally when the actuator is rotated about a central longitudinal axis of the handle assembly. The circular protrusion may be configured to engage one or more recesses of the pair of frame arms to lock the actuator. The tube may include a laser cut pattern with a first degree of pitch in a proximal section of the tube and a second degree of pitch in a distal section of the tube, wherein the second degree of pitch is different from the first degree of pitch, and wherein the interior body includes a slot aligned with the distal section of the tube.

In other aspects, a guidewire assembly for positioning within a body of a patient may include an interior body and a handle assembly. The handle assembly may include a handle body including a proximal section, a distal section spaced from the proximal section, and a pair of frame arms coupled to the proximal section and the distal section; a shaft extending through the handle body and coupled to the interior body, wherein the interior body extends longitudinally through the handle body; and an actuator positioned between the pair of frame arms. Rotation of the actuator about a central longitudinal axis of the handle assembly may be configured to move the interior body proximally or distally relative to the handle assembly.

In other aspects, the guidewire assembly may include one or more of the following features. The actuator may include a helical recess configured to receive a helical protrusion of the shaft. The interior body may include a slot at a distal portion of the interior body, and a distal tip of the interior body is coupled to a distal tip portion of a tube. The handle assembly may further include a distal cap removably coupled to a distalmost end of the handle body; the distal cap may be configured to be coupled to the tube, and the tube may be configured to receive the interior body.

In other aspects, a method of moving a guidewire assembly for positioning within a body of a patient may include (i) moving a shaft of the guidewire assembly distally, wherein the shaft includes a tube and an interior body extending longitudinally through the tube; and (ii) rotating an actuator on a handle of the guidewire assembly about a central longitudinal axis of the handle, wherein the interior body moves distally through the tube when the actuator is rotated. A distal portion of the tube transitions from a curved position to an at least partially straight position as the interior body moves into the distal portion of the tube.

Both the foregoing general description and the following detailed description are exemplary and explanatory only and are not restrictive of the features, as claimed. As used herein, the terms “comprises,” “comprising,” “including,” “having,” or other variations thereof, are intended to cover a non-exclusive inclusion such that a process, method, system, device, article, or apparatus that comprises a list of elements does not include only those elements, but may include other elements not expressly listed or inherent to such a process, method, article, or apparatus. Additionally, the term “exemplary” is used herein in the sense of “example,” rather than “ideal.” As used herein, the terms “about,” “substantially,” and “approximately,” indicate a range of values within +/−5% of the stated value.

### BRIEF DESCRIPTION OF THE FIGURES

The accompanying drawings, which are incorporated in and constitute a part of this specification, illustrate various exemplary embodiments and together with the description, serve to explain the principles of the disclosure.

FIG. 1 illustrates an exemplary guidewire system, according to aspects of this disclosure.

FIGS. 2A-2C illustrate exemplary distal portions of a guidewire system, according to aspects of this disclosure.

FIG. 3 illustrates an exemplary hypotube, according to aspects of this disclosure.

FIG. 4 illustrates an exemplary distal portion of the guidewire system of FIG. 1, according to aspects of this disclosure.

FIG. 5 illustrates an exemplary distal portion of the guidewire system of FIG. 1, according to aspects of this disclosure.

FIGS. 6 and 7 illustrate different views of an exemplary handle assembly of the guidewire system of FIG. 1, according to aspects of this disclosure.

FIG. 8 illustrates a side, cross-sectional view of the handle assembly of FIGS. 6 and 7, according to aspects of this disclosure.

FIG. 9 illustrates an exemplary internal shaft of the handle assembly of FIGS. 6 and 7, according to aspects of this disclosure.

FIG. 10 illustrates a side view of potions of an exemplary hypotube of the guidewire system of FIG. 1, according to aspects of this disclosure.

FIGS. 11A and 11B illustrate perspective and side views of portions of another exemplary guidewire system, according to aspects of this disclosure.

FIGS. 12A, 12B, and 12C illustrate side views of portions of the guidewire system of FIGS. 11A and 11B, according to aspects of this disclosure.

FIG. 13 illustrates a perspective view of a distal portion of the guidewire system of FIGS. 11A and 11B with various representative notations illustrating the movement of the distal portion, according to aspects of this disclosure.

### DETAILED DESCRIPTION

Examples of the present disclosure include systems, devices, and methods to improve the efficacy and safety of minimally-invasive surgeries and other medical procedures. For example, aspects of the present disclosure may relate to medical systems, devices, and methods for delivering a medical device to a portion of a patient's inner anatomy, such as, for example, a procedure to remove kidney stones or other material from a patient's kidney or other organ. In some embodiments, the medical systems of the present disclosure may include a guidewire to deliver a medical tool for diagnosis or treatment of a bodily orifice. The medical devices of the present disclosure include guidewires used to assist in the placement of catheters or other medical devices in body lumens. In particularly, the guidewires of the present disclosure may transition between a pre-shaped configuration of a distal portion of the guidewire to a straight configuration of the distal portion of the guidewire.

Embodiments of the present disclosure are described herein in reference to steerable guidewires for use in minimally invasive medical procedures and/or other medical procedures. For example, it is appreciated that aspects of the present invention can be readily adapted for purposes such as, but not limited to, endoscopic retrograde cholangiopancreatography (ERCP), Percutaneous nephrolithotomy (PCNL), balloon and laser angioplasty, nephrostomy, electrode placement, etc. These applications can all benefit from maneuvering a guidewire to a remote site located internal to the patient's body.

Reference will now be made in detail to examples of the present disclosure described above and illustrated in the accompanying drawings. Wherever possible, the same reference numbers will be used throughout the drawings to refer to the same or like parts.

The terms “proximal” and “distal” are used herein to refer to the relative positions of the components of an exemplary medical device. When used herein, “proximal” refers to a position relatively closer to an operator using the medical device. In contrast, “distal” refers to a position relatively farther away from the operator using the medical device. The proximal and distal directions are labeled throughout the figures.

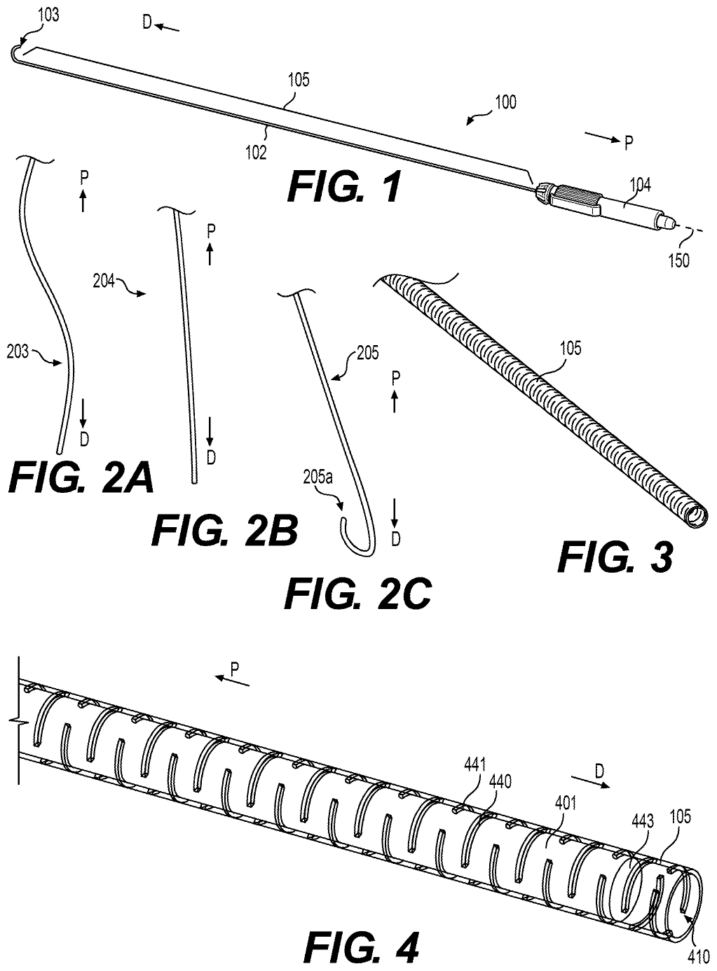

FIG. 1 illustrates a perspective view of a guidewire system 100 including a handle assembly 104, a shaft 102, and a distal portion 103. Shaft 102 may include a tube 105, and, as shown in FIG. 4, an interior body 401 may extend through tube 105. Tube 105 may be coupled to a distal end of handle assembly 104, and distal portion 103 may include a distalmost portion of tube 105. Distal portion 103 may comprise a pre-shaped portion of tube 105, and may comprise nitinol and/or any other shape memory material that has the ability to restore the original shape after deformation.

FIGS. 2A-2C illustrate examples of distal portion 103 of guidewire system 100. FIG. 2A illustrates an S-shaped curve distal portion 203, which may be pre-shaped as an S-shaped curved tube. FIG. 2B illustrates a straight distal portion 204, which may be pre-formed in a straight configuration. FIG. 2C illustrates a curved distal portion 205, which may curve approximately 180-degrees such that the distalmost end 205a of distal portion 205 faces substantially proximally. Although not depicted in the figures, other shapes of distal portion 103 may be incorporated into guidewire system, such as a helical shaped distal portion, a saw-toothed shaped a distal portion, a square-waveform shaped distal portion, a ramp-shaped distal portion, or any other pre-shaped configuration of a tubular body.

FIG. 3 illustrates a portion of tube 105 removed from handle assembly 104. Tube 105 may include a laser cut pattern to provide flexibility at one or more portions of tube 105, such as at a distal portion of tube 105, and geometry or shape of the articulated section of tube 105. As will be discussed further in relation to FIG. 10, tube 105 may include a variable pitch laser cut pattern, which may provide variable stiffness of tube 105 along the longitudinal length of tube 105. The laser cut pattern in tube 105 may include a series of recesses 440, 441 (FIG. 4) in the radially-outer surface, relative to central longitudinal axis 150, along the longitudinal length of tube 105. In some examples, the laser cut pattern may provide a means to maintain articulation within a desired plane, for example the laser cut pattern may prevent movement in specific planes and may limit movement in only one particular plane.

FIG. 4 illustrates a perspective view of a portion of tube 105 and interior body 401. Tube 105 is shown as transparent in FIG. 4 to show interior body 401 positioned within tube 105. Interior body 401 may be cylindrical and may be biased towards a straight configuration. In some examples, interior body 401 may be a wire (e.g., a guidewire). Interior body 401 may be positioned within a central lumen 410 of tube 105, and may be configured to move proximally and distally through lumen 410. Interior body 401 may be sufficiently rigid to move tube 105 from (i) a first position in which tube 105 is bent or otherwise curved without interior body 401 positioned within tube 105 (e.g., as shown in FIGS. 2A and 2C), to (ii) a second position in which tube 105 is substantially or approximately straight with interior body 401 positioned within tube lumen 410 of tube 105. Interior body 401 may include a substantially planar distal front face 443, and distal front face 443 may be configured to be atraumatic when abutting tissue. A proximal end (not shown) of interior body 401 may be coupled to a portion of handle assembly 104, and handle assembly 104 may control the proximal and distal movement of interior body 401 within tube 105.

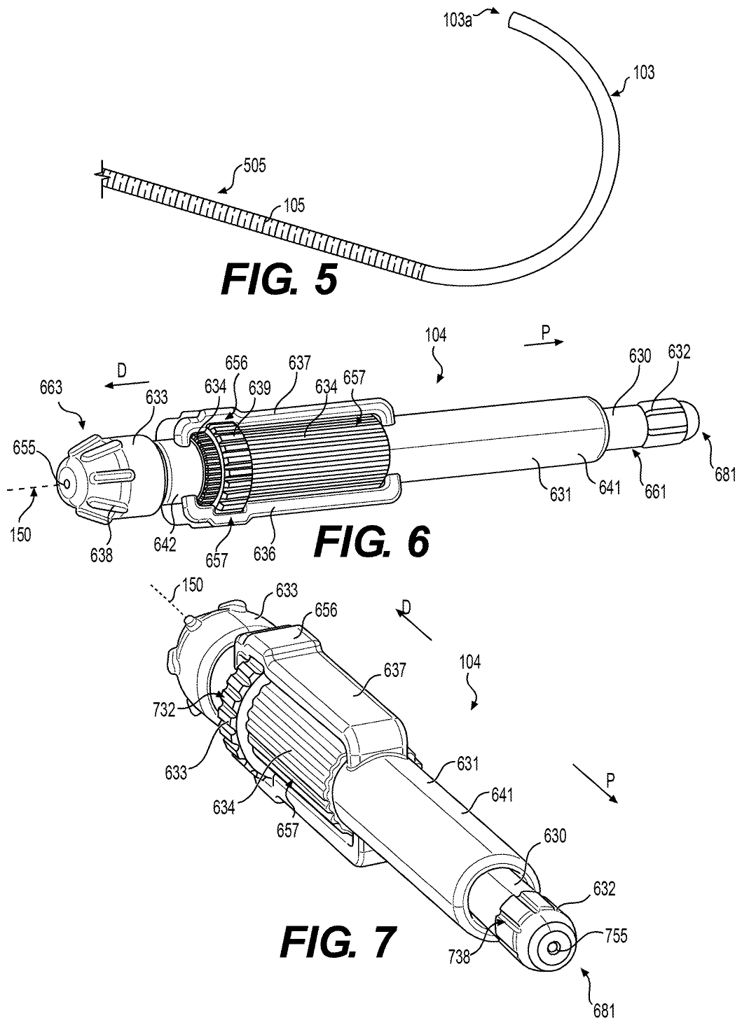

FIG. 5 illustrates an exemplary portion of guidewire system 100 including tube 105 with distal portion 103 and a proximal portion 505. As shown in FIG. 5, proximal portion 505 includes a laser cut pattern of recesses in tube 105, and the laser cut pattern of recesses in tube 105 may be configured to increase the flexibility of tube 105. Distal portion 103 has a pre-shaped curve of approximately 180-degrees, or a bias towards a U-shaped configuration. Distalmost end face 103a of distal portion 103 may face the proximal direction. Distal portion 103 does not include the laser cut pattern of the proximal portion 505 of tube 105. When interior body 401 (FIG. 4) is pushed distally through tube 105 of FIG. 5, distal portion 103 will transition from a U-shaped configuration (shown in FIG. 5) to a substantially or approximately straight configuration when interior body 401 is positioned within distal portion 103 across the entire length of distal portion 103. When interior body 401 is removed from within distal portion 103 of FIG. 5 (i.e., proximally retracted), distal portion 103 will transition to the U-shaped configuration shown in FIG. 5. As will be discussed further herein below, a user may actuate an actuator of handle assembly 104 to move interior body 401 distally or proximally through tube 105, for example, into and out of distal portion 103.

FIGS. 6 and 7 illustrate perspective views of handle assembly 104 of FIG. 1 with shaft 102 removed from or otherwise uncoupled from handle assembly 104. As shown in FIG. 6, handle assembly 104 may include a handle body 631, an actuator 634, a shaft 630, a distal cap 633, and a proximal cap 632. Handle body 631 may be substantially cylindrical, and a proximal portion 641 of handle body 631 may include a circular radially-outer surface, relative to a central longitudinal axis 150 of handle assembly 104. Handle body 631 may include a pair of frame arms 636, 637 connecting proximal portion 641 to a distal portion 642 of handle body 631. Each frame arm 636, 637 may extend around actuator 634, may have a rectangular cross-section taken perpendicular to central longitudinal axis 150, and may include a protruding portion 656, 657 extending radially-outward relative to central longitudinal axis 150. Each protruding portion 656, 657 may be configured to receive a circular protrusion 639 of actuator 634. A first frame arm 636 of the pair of frame arms 636, 637 may be positioned at an opposite side of central longitudinal axis 150 from a second frame arm 637 of the pair of frame arms 636, 637. Distal portion 642 of handle body 631 may extend distally beyond the distalmost end of each frame arm 636, 637. As discussed in detail below, distal cap 633 may be removably coupled to distal portion 642 of handle body 631, and proximal cap 632 may be removably coupled to a proximal end 661 of shaft 630.

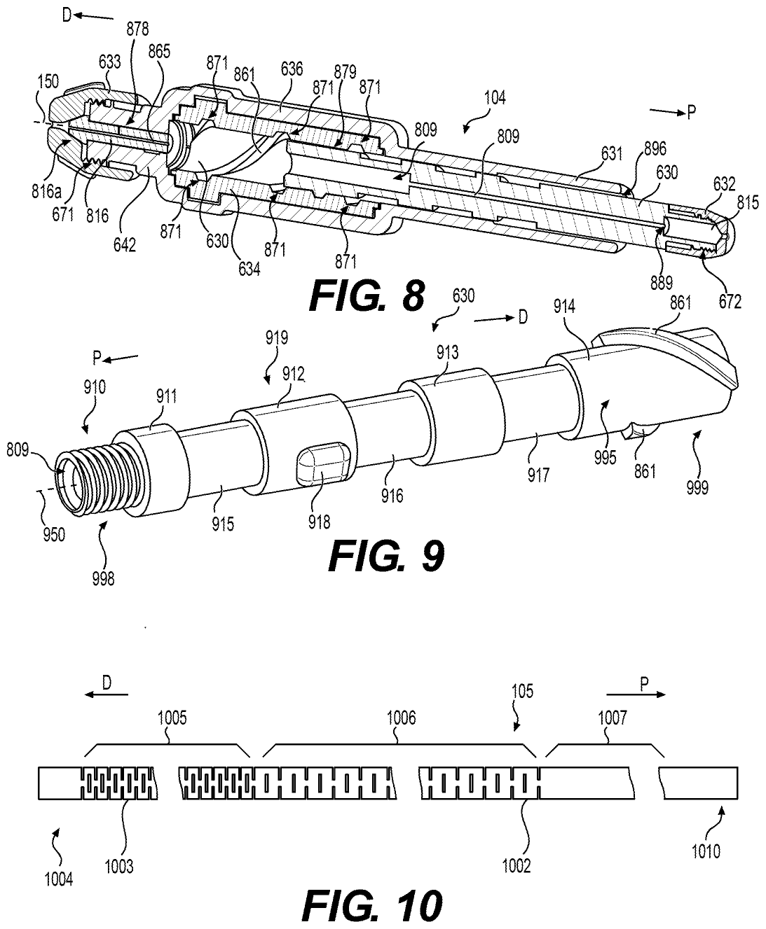

Distal cap 633 may be cylindrical and may include a conical distal portion 663. Distal cap 633 may taper (i.e., from a proximal portion to a distal portion) from a first circumference about central longitudinal axis 150 to a second circumference about central longitudinal axis 150 smaller than the first circumference. A central lumen 655 may extending longitudinally through the center (i.e., the radial center) of distal cap 633, and central lumen 655 may be configured to receive a portion of interior body 401. Threads 671 (shown in FIG. 8) may be positioned within central lumen 655, and threads 671 may be configured to removably couple distal cap 633 to handle body 631 (i.e., to distal portion 642). Distal cap 633 may include a series of protrusions 638 protruding radially-outward, relative to central longitudinal axis 150, from conical distal portion 663. Each protrusion 638 may be substantially oval-shaped and may extend longitudinally in the proximal-distal direction across conical distal portion 663. The series of protrusions 638 may facilitate gripping of distal cap 633, for example, when coupling and/or uncoupling distal cap 633 to handle body 631.

Proximal cap 632 may be cylindrically shaped and may include a rounded proximal end 681. A central lumen 755 of proximal cap 632 may extend longitudinally through proximal cap 632 and may be aligned with central longitudinal axis 150. Central lumen 755 may be configured to receive a portion of interior body 401. In some examples, interior body 401 may extend entirely through proximal cap 632 and a proximalmost portion of interior body 301 may be positioned outside of proximal cap 632 and shaft 630. Proximal cap 632 may include longitudinal recesses 738 extending longitudinally on a radially-outer surface, relative to central longitudinal axis 150, of proximal cap 632. Recesses 738 may facilitate gripping of proximal cap 632, for example, when coupling and/or uncoupling proximal cap 631 to handle body 631. Threads 672 (FIG. 8) may be positioned within proximal cap 632 and may be configured to removably couple proximal cap 632 to shaft 630, and shaft 630 may have corresponding threading to couple proximal cap 632 to shaft 630.

Actuator 634 may be cylindrical and may include longitudinal ridges 657 extending longitudinally from a proximal end to a distal end of actuator 634. Longitudinal ridges 657 may facilitate a user's grip on actuator 634, for example, to rotate actuator 634 clockwise or counterclockwise. A circular protrusion 639 may extend circumferentially around actuator 634, and circular protrusion 639 may include longitudinal ridges 732 configured to facilitate gripping circular protrusion 639 for rotating clockwise or counterclockwise about axis 150. Circular protrusion 639 may be aligned with and adjacent to protrusions 656, 657 of each frame arm 636, 637. Actuator 634 may be a knob, roller actuator, or any other actuator. Actuator 634 may be configured to rotate about central longitudinal axis 150 relative to handle body 631, for example, both clockwise and counterclockwise.

Referring to the side, cross-sectional view of handle assembly 104 shown in FIG. 8, actuator 634 includes a lumen 879 extending longitudinally through actuator 634. A radially-inward facing surface 879 forming lumen 879 may be cylindrical and may include one or more helical recesses 871 extending circumferentially along an inner circumferential surface about central longitudinal axis 150. Helical recesses 871 of actuator 634 may be configured to receive one or more protrusions 861 of shaft 630 (FIG. 9). As shown in FIG. 8, lumen 879 is configured to receive shaft 630

A proximal collet 815 may be positioned at a proximalmost end of shaft 630. Note proximal collet 815 is now shown in cross-section in FIG. 8. Proximal collet 815 may be cylindrical and may include a conical proximalmost end portion. Proximal collet 815 may be configured to receive a portion of interior body 401, and proximal collet 815 may be configured to retract radially-inward and couple to interior body 401 when proximal cap 632 is coupled to shaft 630. Proximal collet 815 may help to fixedly couple interior body 401 to a proximal portion of shaft 630, such that proximal and distal movement of shaft 630 moves interior body 401 proximally or distally, respectively. Proximal collet 815 may be received within a recessed portion of shaft 630 at a proximalmost end of shaft 630, and proximal collet 815 may be longitudinally aligned with central longitudinal lumen 809 of shaft 630. In some examples, proximal collet 815 may include a longitudinal lumen extending through proximal collet 815 configured to receive a proximal portion of interior body 401, and the longitudinal lumen of proximal collet 815 may extend the entire length of proximal collet 815 from a proximalmost end to a distalmost end of proximal collet 815.

A distal collet 816 may be positioned within distal portion 642 of handle body 631. Distal collet 816 may be substantially cylindrical and may include a conical-shaped distalmost end 816a. Distal collet 816 may be configured to receive interior body 401 in an internal channel 865 of distal collet 816. Internal channel 865 may be configured to allow interior body 401 to move proximally or distally (i.e., longitudinally) through internal channel 865, and restrict lateral or radial movement of internal body 401. Internal channel 865 may extending longitudinally through distal collet 816 and may be longitudinally aligned with central longitudinal axis 150. Distal collet 816 may be received by handle body 631 within a channel 878 of distal portion 642. Distal collet 816 and distal cap 633 are configured to couple tube 105 to a distal end of handle assembly 104. Tube 105 may be clamped between distal collet 816 and distal cap 633, and lumen 410 of tube 105 may be longitudinally aligned with central longitudinal axis 150 when tube 105 is coupled to distal collet 816 and distal cap 633. Distal cap 633 may include a conical interior shape configured to correspond to the conical distalmost end 816a of distal collet 816, which may help secure the proximal end of tube 105 when clamped between distal collet 816 and distal cap 633.

As shown in FIG. 8, helical recess(es) 871 of actuator 634 receives helical protrusions 861 of shaft 630. A distal portion of shaft 630 is not shown in cross-section to illustrate helical protrusion 861 received within helical recess 871. Helical protrusions 861 may be positioned at a distal portion of shaft 630. Shaft 630 may be configured to move proximally or distally relative to actuator 634 and handle body 631. Shaft 630 may be cylindrical and may be positioned within handle body 631 and may extend proximally from a proximal end of handle body 631. Shaft 630 may include a lumen 809 extending longitudinally through shaft 630, and lumen 809 may be configured to receive interior body 401. Lumen 809 may extend the entire length of shaft 630 from an opening in the proximalmost end of shaft 630 to an opening in the distalmost end of shaft 630. Lumen 809 may be aligned with lumen 865 of distal collet 816, and lumen 809 may also be aligned with a lumen 889 of proximal collet 632. When interior body 401 is coupled to a proximal end of shaft 630 via proximal collet 815 and proximal cap 632, interior body 401 may move proximally or distally as shaft 630 moves proximally or distally, respectively.

FIG. 9 illustrates a perspective view of shaft 630 removed from handle assembly 104. Note the proximal and distal directions in FIG. 9 are opposite from FIG. 8 (as shown via the proximal (P) and distal (D) arrows in each figure). Shaft 630 may include a proximal end portion 998 and a distal end portion 999. Threads 910 configured to receive proximal cap 632 may be positioned at proximal end portion 998 of shaft 630. A series of protruding portions 911, 912, 913, 914 may be longitudinally spaced from each other along the length of shaft 630, and a series of recessed portions 915, 916, 917 may be positioned between pairs of the protruding portions 911, 912, 913, 914. A distalmost protruding portion 914 may be cylindrical and may include one or more helical protrusions 861 extending across a radially-outer surface 995, relative to a central longitudinal axis 950 of shaft 630, of protruding portion 914. Distalmost protruding portion 914 may extend to a distalmost end of shaft 630. Helical protrusion(s) 861 may extend from the distalmost end of shaft 630 (i.e., a distalmost end of distalmost protruding portion 914) to a proximal portion of protruding portion 914. In some aspects, distalmost protruding portion 914 may extends proximally of a distal end of helical protrusion(s) 861. One or more protrusions 918, 919 may extend radially outward, relative to longitudinal axis 950, from protruding portion 912. Protrusion 919 is not shown in the figures and may be positioned on a laterally opposite side of protruding portion 912. Each of protrusions 918, 919 may support shaft 630 and facilitate positioning shaft 630 within a central channel 896 of handle body 631. For example, protrusions 918, 919 may position shaft 630 within a central portion of channel 896 and may prevent lateral movement, or movement transverse to longitudinal axis 150. In some examples, protrusions 918, 919 may be rubber to provide friction between shaft 630 and a radially-inward facing surface, relative to axis 150, of channel 896 of handle body 631. When protrusions 918, 919 are rubber or another material configured to provide a friction engagement with an interior surface of handle body 631, actuator 634 may be released and the friction engagement between protrusions 918, 919 and an interior surface of handle body 631 may prevent shaft 630 from rotating about axis 150, which may allow a user to release actuator 634 and maintain the current position of distal portion 103. Any number of protruding portions 911, 912, 913, 914 and recessed portions 915, 916, 917 may be included in shaft 630.

FIG. 10 illustrates a side view of an exemplary tube 105 of guidewire system 100 with a proximal end 1010 and a distal end 1004. Portions of tube 105 have been removed in FIG. 10 for illustration purposes. Tube 105 may include a laser cut pattern with a proximal section 1006 and a distal section 1005. Proximal section 1006 may have a higher pitch than distal section 1005. Proximal section 1006 with the higher pitch may facilitate pushability of tube 105, and the lower pitch of distal section 1005 may increase the flexibility of the distal section 1005. Tube 105 may also include a proximal portion 1007, for example, without any laser cut pattern.

During use, guidewire assembly 100, specifically shaft 102, may be introduced into a body cavity, orifice, or incision of a patient. The user may then move shaft 102 into a body lumen, for example, navigating using handle assembly 104 to move distal portion 103 of shaft 102. The user may manipulate shaft 102 such that distal portion 103 of shaft 102 is proximate to target tissue, object, site, etc., such as a kidney stone, for example. To move distal portion 103, the user may rotate actuator 634 about central longitudinal axis 150 of handle assembly 104. As actuator 634 rotates about axis 150, helical recess(es) 871 of actuator 634 engages helical protrusion(s) 861 of shaft 630 to move shaft 630 proximally or distally relative to handle body 631. For example, a user may rotate actuator 634 counter-clockwise to distally advance the interior body 401, which may move shaft 102 distally. By moving shaft 630 distally, interior body 401 will be advanced distally through tube 105 and into distal portion 103 to at least partially straighten distal portion 103. A user may rotate actuator 637 clockwise, for example, to move shaft 630 proximally through handle body 631 and pull interior body 401 proximally through tube 105. As interior body 401 moves proximally out of distal portion 103 of tube 105, distal portion 103 will transition from an at least partially straight configuration to a pre-shaped configuration, such as one of the pre-shaped configurations shown in FIGS. 2A-2C. By enabling the user to transition distal portion 103 between a straight and a pre-shaped configuration, a user may more easily navigate through torturous body cavities because of the plurality of different shapes distal portion 103 of tube 105 may transition between. A user may also push shaft 102 distally or pull shaft 102 proximally to help reposition shaft 102 within the body of the patient.

In some examples, a user may position distal portion 103 of tube 105 at a target tissue, object, site, etc., within a patient, and then remove internal body 401 from guidewire assembly 100 by unscrewing proximal cap 632 from shaft 630 and pulling internal body 401 proximally out of tube 105 and handle assembly 104. Once internal body 401 is removed from guidewire assembly 100, the user may then uncouple handle assembly 104 from tube 105 by unscrewing distal cap 633 and pulling tube 105 distally out of distal cap 633. Once internal body 401 and handle assembly 104 are removed, a user may apply a contrast liquid, such as a contrast agent or other dye or liquid coloring (e.g. iodine-based contrast media or any other contrast agent known in the art) to the proximal end of tube 105 to apply the contrast agent to the target anatomy. In some examples, after removal of internal body 443, a user may apply a contrast liquid through proximal collet 815 and through lumen 809 of shaft 630 within handle 104, avoiding separating the handle 104 from the tube 105. The contrast agent may help the user to visualize the target anatomy using one or more medical imaging apparatuses. A user may then re-couple tube 105 to handle assembly 104, for example, using distal cap 633 and distal collet 816. Once tube 105 is re-coupled to handle assembly 104, the user may insert interior body 401 into proximal collet 815 through shaft 630 and tube 105, and fixedly couple a proximal end of interior body 401 to a proximal end of shaft 630 by tightening proximal cap 632. Since shaft 105 is removable from handle assembly 104, a user may use different shafts 105 (and different tubes 105 and pre-shaped distal portions 103, 203, 204, 205) with the same handle assembly 104.

In some examples, actuator 634 may be configured to lock and hold interior body in position such that a user may release actuator 634, and actuator 634 may maintain its position (e.g., helping to prevent interior body from moving proximally or distally) without further rotating about central longitudinal axis 150. For example, circular protrusion 639 of actuator 634 may be rubber or coated in rubber, and circular protrusion 639 may engage (i.e., frictionally engage) recesses 656, 657 of frame arms 636, 637 to help prevent rotation of actuator 634 when a user releases actuator 634. Longitudinal ridges 732 of circular protrusion 639 may increase engagement and friction between circular protrusion 639 and recesses 656, 657, and may provide a ratcheting mechanism to help secure actuator 634 at different positions. In some examples, actuator 634 and/or circular protrusion 639 may be coated with any other material that provides frictional engagement between actuator 634 and recesses 656, 657 of frame arms 636, 637.

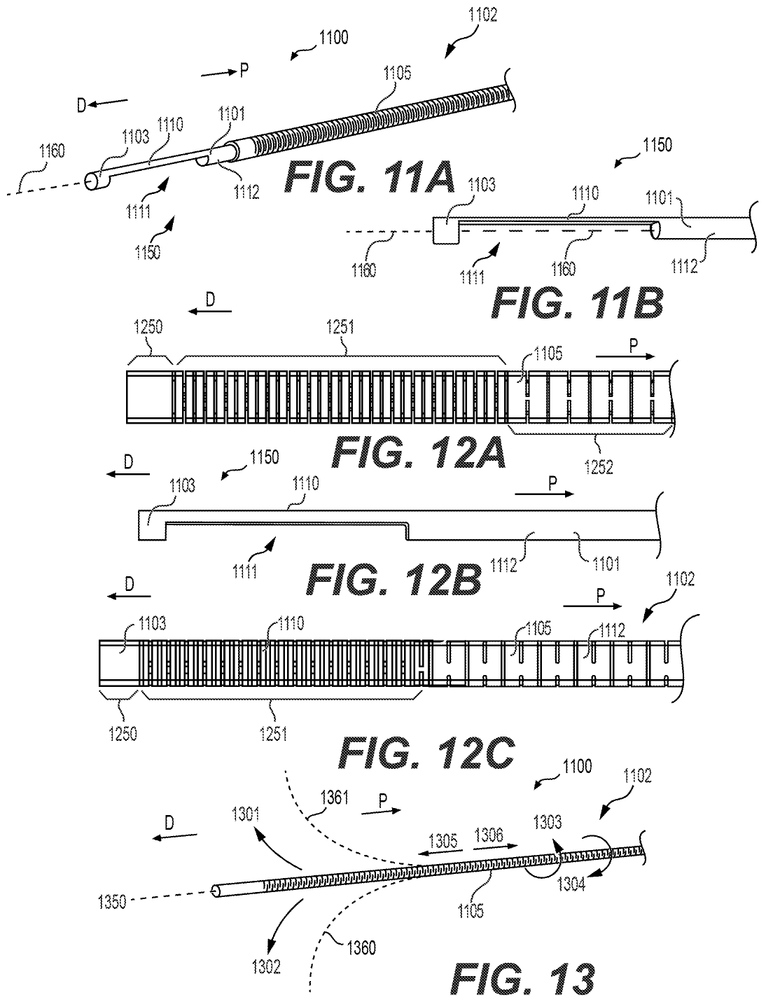

FIGS. 11A-13 illustrate distal portions of various components of an alternative guidewire system 1100. Alternative guidewire system 1100 may include any of the structure and properties of guidewire system 100, and may include a handle assembly 104 and a shaft 1102 coupled to handle assembly 104 in the same manner as described hereinabove in relation to guidewire assembly 100. As shown in FIG. 11A, shaft 1102 may include an interior body 1101 and a tube 1105. Interior body 1101 may be positioned within a channel of tube 1105, and may be configured to move proximally and distally through tube 1105. Shaft 1102 is shown partially disassembled in FIG. 11A for illustration purposes, with interior body 1101 extending distally out of tube 1105.

Interior body 1101 may be cylindrical and include any of the features of interior body 401. Interior body 1101 may include a distal portion 1150 including a distal tip 1103 and a recessed portion 1110 forming a slot 1111 that faces radially outward to a side of interior body 1101. A proximal portion 1112 of interior body 1101 may extend from distal portion 1150 to a handle assembly of guidewire assembly 1100. Distal tip 1103 may be cylindrical and may be configured to abut and/or couple to a distal end portion of tube 1105. Slot 1111 may be an eccentric cut out of interior body 1101, with a central longitudinal axis 1160 extending longitudinally through slot 1111. Recessed portion 1110 may be spaced from central longitudinal axis 1160. In some examples, recessed portion 1110 may include a flat surface facing central longitudinal axis 1160 and facing radially outward to a side of interior body 1101. Recessed portion 1110 may include a series of cuts and/or recesses (not shown) configured to increase the flexibility of recessed portion 1110. In some examples, proximal portion 1112 may be cylindrical, and in other examples proximal portion 1112 may be rectangular (not shown). Since slot 1111 is an eccentric cutout of interior body 1101, slot 1111 may be configured to bend in a first direction when interior body 1101 is pushed distally relative to tube 1105 and distal tip 1103 is coupled to tube 1105, and configured to bend in a second direction (opposite from the first direction) when interior body 1101 is pulled proximally relative to tube 1105 and distal tip 1103 is coupled to tube 1105. In some examples, the first direction is a direction extending from axis 1160 towards recessed portion 1110, and the second direction is a direction extending from axis 1160 away from recessed portion 1110. FIG. 11B illustrates a side view of distal portion 1150 of interior body 1101 including distal tip 1103, recessed portion 1110, proximal portion 1112, and slot 1111. FIG. 11B illustrates how central longitudinal axis 1160 extends through slot 1111. In some examples, interior body 1101 may be partially or entirely made of nitinol and/or any other shape memory material that has the ability to restore the original shape after deformation.

FIG. 12A illustrates a side view of a distal portion of tube 1105. Tube 1105 may include a distal tip portion 1250, a flexible section 1251, and a relatively rigid section 1252. The flexible section 1251 may extend longitudinally between and connect distal tip portion 1250 and rigid section 1252. Tube 1105 may include a laser cut pattern, and the laser cut pattern on the rigid section 1252 may have a higher pitch than the laser cut pattern on the flexible section 1251. For example, slots or openings of the laser cut pattern may be longitudinally spaced further apart in rigid section 1252 compared to the flexible section 1251. Distal tip portion 1250 may not include a laser cut pattern. It is understood that rigid section 1252 is flexible, but is less flexible than flexible section 1251.

FIG. 12B illustrates a side view of distal portion 1150 of interior body 1101. Slot 1111 of interior body 1101 may be configured to align with flexible section 1251, distal tip 1103 may be configured to align with distal tip portion 1250, and proximal portion 1112 may be configured to align with rigid portion 1252. As shown, slot 1111 may be shorter than flexible section 1251.

FIG. 12C illustrates shaft 1102 in a fully assembled state with interior body 1101 coupled to tube 1105. Distal tip 1103 of interior body 1101 may be coupled to distal tip portion 1250 of tube 1105. Recessed portion 1110 and proximal portion 1112 may be configured to move within tube 1105 when in a fully assembled state. Distal tip 1103 may be laser welded to distal tip portion 1250. In some examples, recessed portion 1110 may extend through flexible section 1251, and proximal portion 1112 may extend through rigid portion 1252.

When a user actuates the handle assembly of guidewire assembly 1100, interior body 1101 may move proximally or distally through tube 1105 to a distal portion of tube 1105, for example distal tip 1250, flexible section 1251, and/or rigid section 1252. Since distal tip 1103 is coupled to distal tip portion 1250, relative movement occurs between (i) recessed portion 1110 and proximal portion 1112 and (ii) flexible portion 1251 and rigid portion 1252 of tube 1105. FIG. 13 illustrates a perspective view of a distal portion of guidewire assembly 1100 including various directional arrows to show the movement capabilities of shaft 1102. A user may move shaft 1102 proximally 1305 or distally 1306, may rotate shaft 1102 about central longitudinal axis 1350 in a clockwise 1303 or counterclockwise 1304 direction, and may bend a distal portion of shaft upward 1301 or downward 1302. Although shown as upward 1301 and downward 1302 in FIG. 13, the bi-directional bending capability of shaft 1102 may be any first direction 1301 and any second direction 1302 opposite from the first direction 1301. Representative dotted lines 1360 and 1361 illustrate an example of a position of shaft 1105 in a bent downward position 1360 and an example of a position of shaft 1105 in a bent upward position 1361.

Slot 1111 in interior body 1101 allows body 1101 at recessed portion 1110 to be more flexible than proximal portion 1112 of interior body 1101. This difference in flexibility along the longitudinal length of interior body 1101 allows interior body 1101 to bend tube 1105 in the manner shown in FIG. 13. When a user pushes interior body 1101 distally relative to shaft 1105 (via handle assembly), interior body 1101 proximal of tip 1103 may move through tube 1105, recessed portion 1110 (and, in some examples, a distal portion of proximal portion 1112) may bend in a first direction, and distal tip 1103 may be prevented from moving distally relative to tube 1105 because distal tip 1103 is coupled to distal portion 1250. Since recessed portion 1110 bends when interior body 1101 is moved distally, interior body bends tube 1105 upward 1301 or downward 1302. Since slot 1111 is an eccentric cut in interior body 1101, when a user moves interior body proximally relative to shaft 1105, recessed portion 1110 may bend in the opposite direction as when interior body 1101 is moved distally. By providing slot 1111, interior body 1101 will consistently bend upward 1301 and downward 1302 (as compared to other directions) when interior body 1101 is moved distally and proximally within tube 1105.

The disclosed guidewire assemblies 100, 1100 and portions thereof shown in the figures and discussed above facilitate positioning of other medical devices during a medical procedure. The guidewire assemblies 100, 1100 and portions thereof may help enable efficient and effective procedures by facilitating movement of shaft 102, 1102 through patient anatomy and maintaining guidewire positioning at target anatomy regions, while also providing a conduit to apply a contrast agent to a target anatomy. The design of handle assembly 104 may facilitate movement of shaft 102, 1102 through patient anatomy, which may decrease procedure time and limit procedural error and/or patient complications.

It is contemplated that the guidewires, systems, and methods discussed herein may be applicable to any endoscopic and/or minimally invasive procedure. For example, the systems, devices, and methods discussed above may be used during a percutaneous nephrolithotomy/nephrolithotripsy (PCNL), endoscopic retrograde cholangiopancreatography (ERCP), balloon and laser angioplasty, nephrostomy, electrode placement, etc. The systems, devices, and methods discussed above may also be used in procedures to remove ureteral stones, gallstones, bile duct stones, polyps, stent placement, gastroenteral anastomosis, choledochoduodenostomy, etc.

While principles of the present disclosure are described herein with reference to illustrative examples for particular applications, it should be understood that the disclosure is not limited thereto. Those having ordinary skill in the art and access to the teachings provided herein will recognize additional modifications, applications, embodiments, and substitution of equivalents all fall within the scope of the features described herein. Accordingly, the claimed features are not to be considered as limited by the foregoing description.

We claim:

1. A guidewire assembly comprising:
a handle assembly including:
a handle body;
a shaft extending through the handle body; and
an actuator;
a tube extending distally from the handle assembly and including a proximal portion and a distal portion; and
an interior body extending from the handle assembly through the tube to the distal portion of the tube, wherein the interior body is coupled to the shaft;
wherein the shaft is configured to move longitudinally through the handle body as the actuator is rotated to move interior body longitudinally through the tube.

2. The guidewire assembly of claim 1, wherein a distal portion of the interior body is shape-memory material.

3. The guidewire assembly of claim 2, wherein the distal portion of the interior body is U-shaped, S-shaped, helical shaped, spiral shaped, and/or includes a one hundred and eighty degree curve in the interior body.

4. The guidewire assembly of claim 1, wherein the proximal portion of the tube includes a laser cut pattern.

5. The guidewire assembly of claim 1, wherein the tube is coupled to the handle assembly at a distal end of the handle assembly.

6. The guidewire assembly of claim 5, wherein the tube is removably coupled to the distal end of the handle assembly via a distal cap and a distal collet.

7. The guidewire assembly of claim 1, wherein the actuator is cylindrical and extends longitudinally through a central longitudinal axis of the handle body, and wherein the shaft extends longitudinally through a lumen of the actuator.

8. The guidewire assembly of claim 7, wherein the handle body includes a cylindrical proximal portion, a distal portion spaced from the proximal portion, and a pair of frame arms coupled to the distal portion and the proximal portion.

9. The guidewire assembly of claim 1, wherein a distal tip of interior body is coupled to a distal tip portion of the tube.

10. The guidewire assembly of claim 8, wherein the actuator includes a circular protrusion received by a recess of each of the pair of frame arms.

11. The guidewire assembly of claim 1, wherein the interior body is removably coupled to a proximal end of the shaft via a proximal collet and a proximal cap.

12. The guidewire assembly of claim 1, wherein the actuator includes a helical recess configured to receive a helical protrusion of the shaft.

13. The guidewire assembly of claim 12, wherein the actuator is configured to move the shaft proximally or distally when the actuator is rotated about a central longitudinal axis of the handle assembly.

14. The guidewire assembly of claim 10, wherein the circular protrusion is configured to engage one or more recesses of the pair of frame arms to lock the actuator.

15. The guidewire assembly of claim 1, wherein the tube includes a laser cut pattern with a first degree of pitch in a proximal section of the tube and a second degree of pitch in a distal section of the tube, wherein the second degree of pitch is different from the first degree of pitch, and wherein the interior body includes a slot aligned with the distal section of the tube.

16. The guidewire assembly for positioning within a body of a patient, comprising:
an interior body;
a handle assembly, including:
a handle body including a proximal section, a distal section spaced from the proximal section, and a pair of frame arms coupled to the proximal section and the distal section;
a shaft extending through the handle body and coupled to the interior body, wherein the interior body extends longitudinally through the handle body; and
an actuator positioned between the pair of frame arms;
wherein rotation of the actuator about a central longitudinal axis of the handle assembly is configured to move the interior body proximally or distally relative to the handle assembly.

17. The guidewire assembly of claim 16, wherein the actuator includes a helical recess configured to receive a helical protrusion of the shaft.

18. The guidewire assembly of claim 17, wherein the interior body includes a slot at a distal portion of the interior body, and wherein a distal tip of the interior body is coupled to a distal tip portion of a tube.

19. The guidewire assembly of claim 18, wherein the handle assembly further comprises a distal cap removably coupled to a distalmost end of the handle body; wherein the distal cap is configured to be coupled to the tube, and wherein the tube is configured to receive the interior body.

20. A method of moving a guidewire assembly for positioning within a body of a patient, comprising:
moving a shaft of the guidewire assembly distally, wherein the shaft includes a tube and an interior body extending longitudinally through the tube; and
rotating an actuator on a handle of the guidewire assembly about a central longitudinal axis of the handle, wherein the interior body moves distally through the tube when the actuator is rotated;
wherein a distal portion of the tube transitions from a curved position to an at least partially straight position as the interior body moves into the distal portion of the tube.
